(19) United States
(12) Patent Application Publication
(10) Pub. No.: US 2007/0265591 A1
(43) Pub. Date: Nov. 15, 2007

(54) Disposable absorbent article having removable indicium for facilitating properly fitting the article to a wearer's body and a method of use

(75) Inventors: Jeffrey James Loritz, Appleton, WI (US); Julie Ann Paveletzke, Appleton, WI (US); Joseph Andrew Mlinar, Appleton, WI (US); Suzanne Marie Schmoker, Oshkosh, WI (US); Kenneth John Wagner, Greenville, WI (US); James Lawrence Kerscher JR., Appleton, WI (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 11/432,218

(22) Filed: May 11, 2006

(51) Int. Cl. A61F 13/15

(52) U.S. Cl. 604/361; 604/385.3; 604/385.31

(57) ABSTRACT

A disposable absorbent article is disclosed which includes a chassis having an inner surface and an outer surface. The chassis also has a front region and a back region joined together by a pair of side seams to form a unitary article with a waist opening and a pair of leg openings. An absorbent is secured to the inner surface of the chassis. The disposable absorbent article also includes a waistband which extends along at least a portion of the back region proximate the waist opening. The article further includes a removable indicium positioned on the waistband which provides a visual indicator.

### BACKGROUND OF THE INVENTION

Disposable absorbent articles are available in many different sizes, styles and configurations and are designed for absorbing human exudate, such as urine and fecal matter. Various disposable absorbent articles are intended to be worn by infants, toddlers or adults and are designed for single use. By “single use” it is meant that the disposable absorbent article will be disposed of after being used once instead of being laundered or cleaned for reuse, as is typical of regular cloth underwear. Examples of some disposable absorbent articles include but are not limited to infant diapers, child training pants, feminine menstrual pants and adult incontinent undergarments. Many of such disposable absorbent articles are similar in appearance, size and shape to regular cloth underwear except that they are formed from a variety of nonwoven or film materials and normally include an absorbent, as well as elastics. The absorbent allows the disposable absorbent articles to absorb and retain body waste while the elastics permit the disposable absorbent articles to snugly conform to the anatomy of the wearer's torso.

Most manufacturers of such disposable absorbent articles are constantly looking to improve the appearance, aesthetics and normalcy of such articles to simulate regular cloth underwear. One reason for this is that adult users of disposable incontinent undergarments have been preconditioned by years of wearing regular cloth underwear. “Cloth underwear” includes underwear formed from various natural and/or synthetic materials, including cotton, nylon, rayon, SPANDEX, as well as other materials known to those skilled in the art.

Today, many caregivers as well as wearers of such disposable absorbent articles have a difficult time differentiating the front and back regions of such articles. This is especially true for older adults who have become incontinent and are using such articles for a first time. In addition, some older adults have poor eye sight which can add to the confusion of properly positioning the undergarment on their body. Furthermore, many users of such disposable absorbent articles do not want the brand name to remain or appear on the disposable absorbent article during or after use of the article. Physiologically, they do not want anyone to know that they have to or have been wearing such undergarments.

Kimberly-Clark Corporation (K-C) is a manufacturer of many disposable absorbent articles including DEPEND Protective Underwear and POISE Panty. These articles utilize two blue colored elastic strands that serve as a back indicator. The colored elastic strands are permanently secured to the back region, proximate the waist opening, to differentiate the back region from the front region. However, the color thread is sometimes hard for some older adults to see. In addition, some consumers do not understand the purpose of the two colored elastic strands. Furthermore, at least one competitor has started placing color threads in both the front and back regions for aesthetic effects and this has lead to confusion when a user returns to using a DEPEND or POISE product.

Now a disposable absorbent article has been invented which includes a removable indicium for facilitating fitting the disposable absorbent article to a wearer's body. A method of promoting the sale of a disposable absorbent article is also taught which includes associating the article with an indicium printed on a label and removing the label before the article is worn.

### SUMMARY OF THE INVENTION

Briefly, this invention relates to a disposable absorbent article including a chassis having an inner surface and an outer surface. The chassis also has a front region and a back region joined together by a pair of side seams to form a unitary article having a waist opening and a pair of leg openings. An absorbent is secured to the inner surface of the chassis and a waistband is secured to the chassis. The waistband extends along at least a portion of the back region proximate the waist opening. The article further includes a removable indicium positioned on the waistband which provides a visual indicator for facilitating proper fitting of the disposable absorbent article to a wearer's body.

A method of promoting the sale of a disposable absorbent article is also taught which includes associating the article with an indicium printed on a label and removing the label before the disposable absorbent article is worn.

### BRIEF DESCRIPTION OF THE DRAWINGS

FIG. 1 is a perspective view of a disposable absorbent article having a waistband with a removable indicium positioned on the waistband which provides a visual indicator for facilitating properly fitting the article to a wearer's body.

FIG. 2 is a front view of the disposable absorbent article shown in FIG. 1.

FIG. 3 is a cross-sectional view of the disposable absorbent article shown in FIG. 2 taken along line 3-3.

FIG. 4 is an enlarged cross-sectional view of a portion of the disposable absorbent article shown in FIG. 3, depicting an absorbent assembly secured to an inner surface of the chassis.

FIG. 5 is an enlarged cross-sectional view of a portion of the disposable absorbent article shown in FIG. 3 depicting a waistband formed from a laminate onto which a label is removeably attached.

FIG. 6 is a representation of the brand name “DEPEND” printed on a label.

FIG. 7 is a representation of the letters “KC” printed on a label.

FIG. 8 is a cross-sectional view of an alternative embodiment of a disposable absorbent article having a removable label attached to an exterior surface of the back region of the waistband.

FIG. 9 is a cross-sectional view of still another embodiment of a disposable absorbent article having a removable label attached to an exterior surface of the front region of the waistband.

FIG. 10 is a front view of a length of an elastic waistband having a non-elastic area and having a removable indicium positioned on the non-elastic area.

### DETAILED DESCRIPTION

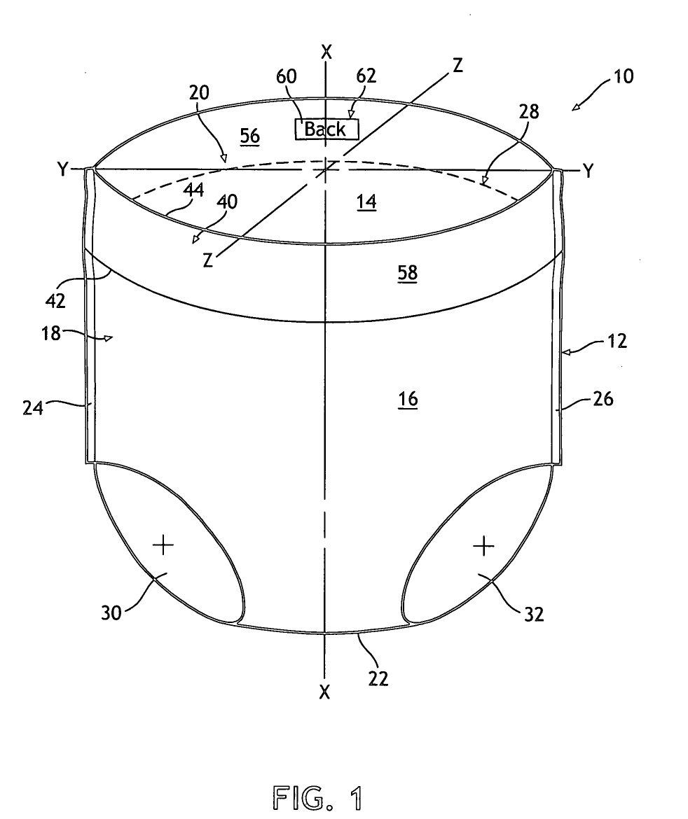

Referring to FIG. 1, a disposable absorbent article 10 is shown which is intended to be worn by people of varying ages, such as infants, toddlers or adults. The disposable absorbent article 10 can be an adult incontinent undergarment, an infant diaper, a child training pant, a feminine menstrual pant, etc. The disposable absorbent article 10 is designed for a single use and is meant to be disposed of after being used once instead of being laundered or cleaned for reuse. The disposable absorbent article 10 is designed to be pulled up around a user's torso without having to first open the undergarment 10 in order to place it on a user's body. In FIG. 1, the disposable absorbent article 10 is shown as it would appear after it has been removed from its package but before it is pulled up around a user's torso.

The disposable absorbent article 10 includes a chassis 12 having an inner surface 14 and an outer surface 16. The chassis 12 also has a front region 18, a back region 20 and a crotch region 22. The crotch region 22 extends between the front and back regions, 18 and 20 respectively, and is situated proximate the groin area of the wearer when the disposable absorbent article 10 is pulled up around the wearer's torso. The front and back regions, 18 and 20 respectively, are joined together by a pair of side seams 24 and 26 to form a unitary article having a waist opening 28 and a pair of leg openings 30 and 32. The side seams 24 and 26 can be formed by a variety of means known to those skilled in the art including but not limited to ultrasonic bonds, adhesive, glue, by a mechanical fastener such as stitching using thread, etc. By a “unitary article” it is meant that the article 10 is designed to be stepped into by a user and the article 10 is then pulled up along the user's legs and thighs and positioned around the user's torso. The unitary article is not presented in an open configuration upon removal from its package. The unitary article does not contain any fastening tabs which have to be secured to another location on the article in order to properly hold the disposable absorbent article 10 around a wearer's torso. In addition, if the pair of side seams 24 and 26 is torn or broken, the unitary article becomes essentially unusable. The disposable absorbent article 10 has a longitudinal central axis X-X, a transverse axis Y-Y and a vertical axis Z-Z.

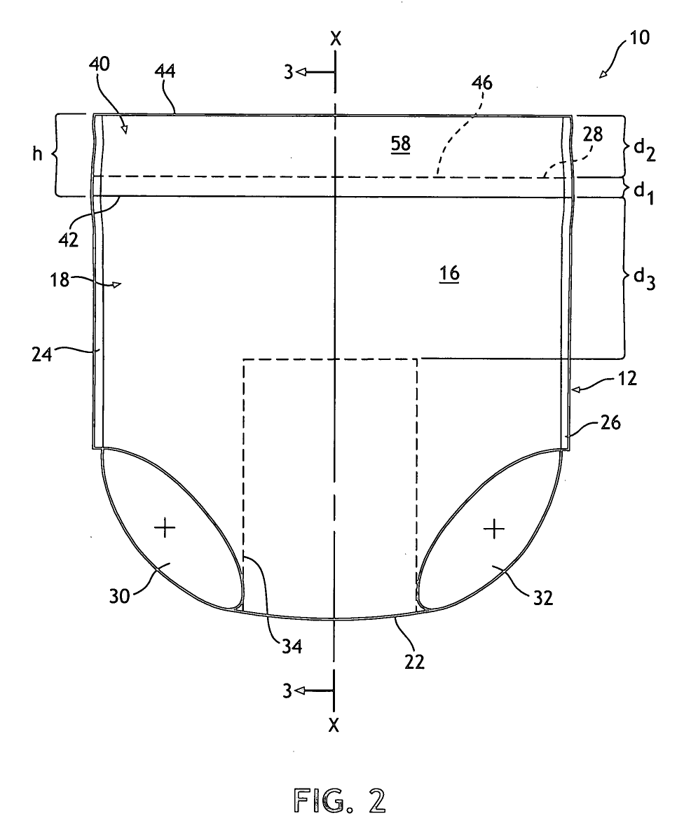

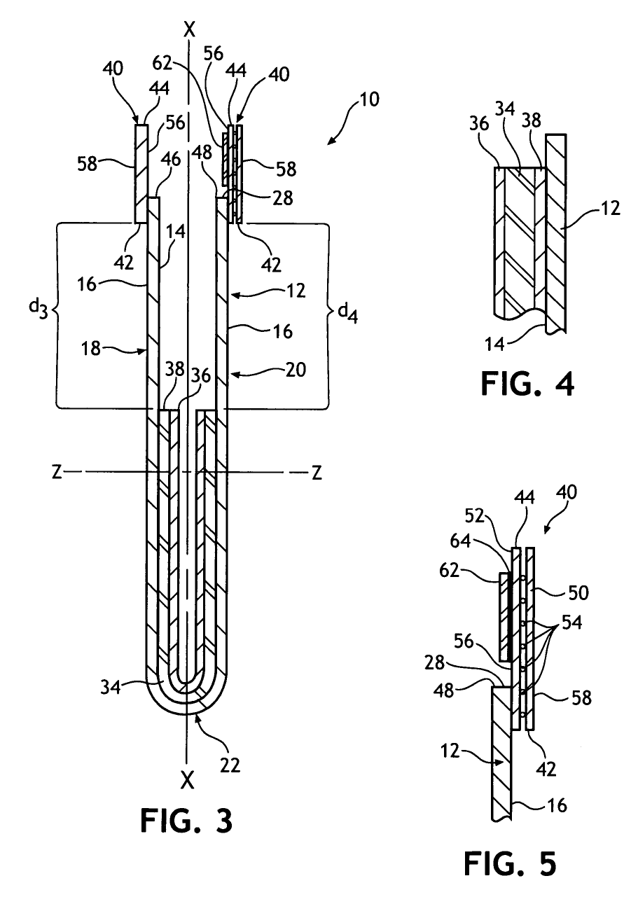

Referring now to FIGS. 2 and 3, the disposable absorbent article 10 also has an absorbent 34 secured to the inner surface 14 of the chassis 12. The absorbent 34 can be secured along a portion of its surface area or over its entire surface area to the inner surface 18 of the chassis 12. The absorbent 34 can be secured by an adhesive (not shown) or by any other means known to those skilled in the art. The absorbent 34 can be attached to at least one of the front, back and crotch regions, 18, 20 and 22 respectively. Alternatively, the absorbent 34 can be attached to at least two of the front, back and crotch regions, 18, 20 and 22 respectively. Desirably, the absorbent 34 can be attached to all three of the front, back and crotch regions, 18, 20 and 22 respectively.

As shown in FIGS. 2 and 3, the absorbent 34 will extend over and desirably cover a major portion of the crotch region 22. By a “major portion” it is meant greater than 50% of the surface area of the crotch region 22. In addition, the absorbent 34 will extend into at least a portion of the front region 18 and/or into a portion of the back region 20. The absorbent 34 can be formed from natural or synthetic materials. The absorbent 34 can be made of air formed, airlaid and/or wet laid composites of fibers. More specifically, the absorbent 34 can be made from cellulosic fibers, wood pulp, textile fibers or from any other absorbent materials known to those skilled in the art. Examples of fibers that can be used include fluff/pulp cellulosic fibers, such as Alliance CR-1654. In addition, any combination of cross-linked pulps, hardwood, softwood and/or synthetic fibers could be utilized. Superabsorbents, typically made from polyacrylic acids, can be combined with the fibers or pulp to increase the absorbent and/or retention capacity of the absorbent 34. The superabsorbents are commonly in solid form and in the shape of small particles, granules, flakes, etc. The superabsorbents can be mixed, combined, attached, printed or otherwise added to the absorbent 34. One such superabsorbent is FAVOR 880 which is commercially available from Stockhausen, Inc., having an office in Greensboro, N.C.

Turning now to FIG. 4, the absorbent 34 can consist of one or more layers of an absorbent material. In FIG. 4, the absorbent 34 is depicted as a single layer of absorbent material. The absorbent 34 can also be partially or completely enclosed or sandwiched between a liquid pervious bodyside liner 36 and a liquid-impervious backsheet 38. A surge layer (not shown) can also be optionally used, which is normally positioned between the bodyside liner 36 and the absorbent 34. The surge layer can function to rapidly acquire and temporarily retain body fluid, such as urine, before it can be absorbed into the absorbent 34. Desirably, the surge layer is also capable of wicking body fluid lengthwise and/or widthwise across its surface as well as directing the body fluid downward in the z-direction, toward the absorbent 34.

Returning to FIGS. 1-3, the disposable absorbent article 10 further includes a waistband 40. The waistband 40 has a first or lower edge 42, a second or upper edge 44 and a height dimension h, (see FIG. 2) measured parallel to the longitudinal central axis X-X. The height dimension h of the waistband 40 can vary from between about 0.5 inches to about 5 inches (about 13 mm to about 127 mm). Desirably, the waistband 40 will have a height dimension h of from between about 0.75 inches to about 3 inches (about 19 mm to about 76 mm). More desirably, the waistband 40 will have a height dimension h of from between about 1 inch to about 2 inches (about 25.4 mm to about 51 mm). Even more desirably, the waistband 40 will have a height dimension h of from between about 1 inch to about 1.5 inches (about 25.4 mm to about 38 mm).

As best depicted in FIG. 3, the waistband 40 is shown being secured to the outer surface 16 of the chassis 12 such that the first or lower edge 42 overlaps a portion of the chassis 12. However, the waistband 40 could be attached to the inner surface 14 of the chassis 12 or be integrally formed within the front and back regions, 18 and 20 respectively. The waistband 40 can extend across the entire width or only along a portion of the width of front region 18. Likewise, the waistband 40 can extend across the entire width or only along a portion of the width of back region 20.

In FIGS. 2 and 3, the front region 18 of the chassis 12 is shown having an upper end 46. In FIG. 3, the back region 20 of the chassis 12 is shown having an upper end 48. These upper ends 46 and 48 represent the waist opening 28 of the disposable absorbent article 10 before the waistband 40 is attached. Stated another way, the waistband 40 can extend above the waist opening 28 of the disposable absorbent article 10.

The waistband 40 can be attached to the chassis 12 in a cantilever fashion such that the first or lower edge 42 overlaps a portion of the chassis 12 and is secured thereto. By “cantilever” it is meant that the waistband 40 is attached and supported by at least one end of the chassis 12 and extends or projects upward from the chassis 12. Another way of stating this is to say that the first or lower edge 42 of the waistband 40 is located below the upper ends 46 and 48 of the chassis 12. In FIG. 2, the overlap between the first or lower edge 42 of the waistband 40 and the upper end 46 of the chassis 12 is designated by a distance d₁. This overlap distance d₁ can vary from between about 0.12 inches to about 2 inches (about 3 mm to about 51 mm). Desirably, the overlap distance d₁ can vary from between about 0.25 inches to about 1.5 inches (about 6 mm to about 38.1 mm). More desirably, the overlap distance d₁ can vary from between about 0.5 inches to about 1 inch (about 12.7 mm to about 25.4 mm). Even more desirably, the overlap distance d₁ can be from between about 0.6 inches to about 0.75 (about 15.2 mm to about 19 mm).

Referring again to FIG. 2, the waistband 40 extends upward from and beyond the upper ends 46 and 48 of the front and back regions, 18 and 20 respectively, by a distance d₂. The distance d₂ should range from between about 0.5 inches to about 4 inches (about 13 mm to about 102 mm). Desirably, the distance d₂will range from between about 0.75 inches to about 2 inches (about 19 mm to about 51 mm). More desirably, the distance d₂will range from between about 1 inch to about 1.5 inches (about 25.4 mm to about 38 mm).

The waistband 40 can be constructed or formed from a variety of materials known to those skilled in the art. Desirably, the waistband 40 is formed from an elastic material having both stretch and retraction capabilities. By “stretch” it is meant that the original length of the elastic material can be elongated or extended to an increased length when subjected to a pulling or tensile force. By “retraction” it is meant that the elongated length of the elastic material can return to or towards its original length after the force used to stretch the material has been removed. An elastic material from which the waistband 40 can be constructed is a laminate consisting of two outer layers of polypropylene spunbond with a plurality of elastic strands sandwiched therebetween. This elastic laminate, as well as other elastic materials, which can be used to form the waistband 40 are known to those skilled in the art. It is also possible to construct the waistband 40 from a laminate which includes one or more elastic strands or filaments sandwiched between two or more outer layers. A stretch bonded laminate (SBL) is one example of an elastic laminate.

Referring now to FIG. 5, the waistband 40 is depicted as a laminate containing a first outer layer 50, a second outer layer 52 and two or more elastic strands 54 positioned therebetween. The first and second outer layers 50 and 52 can be constructed or formed from a woven or a nonwoven material, a natural or synthetic material, an elastic film, a thermoplastic film, or from any other material known to those skilled in the art. The number of elastic strands 54 positioned between the first and second outer layers 50 and 52 can vary depending upon the height dimension h of the waistband 40. For a waistband 40 having a height dimension h of less than about 2.5 inches (about 64 mm), one to twelve elastic strands 54 can be employed. Desirably, the waistband 40 will utilize from between about two to ten elastic strands 54. More desirably, the waistband 40 will utilize from between about two to eight elastic strands 54. In FIG. 5, seven elastic strands 54 are depicted in the waistband 40.

The elastic strands 54 can be formed from LYCRA. The elastic strands 54 can extend along the entire length of the waistband 40. Alternatively, the elastic strands 54 can extend along only a portion of the waistband 40. Furthermore, the elastic strands 54 do not have to be continuous along the entire length of the waistband 40. The elastic strands 54 should be positioned in an approximately parallel relationship to one another but could be arranged in an intersecting or crossing pattern, if desired. The elastic strands 54 should be spaced apart a predetermined distance depending upon how much tension, stretch and retraction is required for a particular product. A spacing distance between two adjacent elastic strands 54 of from between about 5 mm to about 10 mm works well. The exact number of elastic strands 54, the decitex of each elastic strand 54, the exact distance adjacent elastic strands 54 are spaced apart, the method employed for adhering the elastic strands 54 to the outer layers 50 and 52, as well as the stretch, elongation and retraction capabilities of the elastic strands 54, will all dictate how many elastic strands 54 are needed. For example, a diaper having a narrow waistband may not need to have as much staying or holding power to retain the diaper around an infant's torso as a large adult incontinent undergarment designed to hold large quantities of urine voided by the adult user. In addition, an adult wearing such an incontinent undergarment may be more mobile and upright for longer periods of time and therefore require a more secure waistband.

Returning to FIGS. 2 and 3, the waistband 40 is shown as being spaced apart from the absorbent 34. In FIGS. 2 and 3, the waistband 40 is spaced apart from the absorbent 34 in the front region 18 by a distance d₃. In FIG. 3, the waistband 40 is spaced apart from the absorbent 34 in the back region 20 by a distance d₄. The distance d₃ can be less than, equal to or be greater than the distance d₄. The distances d₃ and d₄ can be identical or can vary. The distances d₃ and d₄ should be at least about 0.5 inches (about 12.7 mm). For example, a manufacturer may skew or position the absorbent 34 closer to the waistband 40 in the front region 18 for a male wearer but maintain the absorbent 34 at an equal distance from the waistband 40 in the front and back regions, 18 and 20 respectively, for a female wearer. The reason for this is that the anatomical features of a male user will cause him to insult the disposable absorbent article 10 at a location closer to the front waistband 40 than will a female user.

By locating the waistband 40 away from the absorbent 34 by a distance of at least 0.5 inches (about 12.7 mm) one can be assured that the absorbent 34 will not interfere with the stretching and retraction properties of the waistband 40. This is important for it will allow the waistband 40 to perform its intended function of securely holding and retaining the disposable absorbent article 10 around the wearer's torso even when a large quantity of body fluid, such as urine, has insulted the disposable absorbent article 10.

Referring again to FIG. 1, the waistband 40 is shown being secured completely around the circumference of the waist opening 28. As depicted in FIG. 1, the pair of side seams 24 and 26 can extend up into the waistband 40. Desirably, the pair of side seams 24 and 26 will extend through the entire height dimension h of the waistband 40. More desirably, the waistband 40, including the pair of side seams 24 and 26, will extend 360 degrees around the waist opening 28. Optionally, the waistband 40 can extend along only a portion of the front region 18, proximate the waist opening 28. It is also possible to secure the waistband 40 such that it extends along only a portion of the back region 20, proximate the waist opening 28. Furthermore, it is possible to secure the waistband 40 such that it extends along a portion of both the front and back regions, 18 and 20 respectively, but does not extend 360 degrees around the waist opening 28.

It should also be noted that even though the waistband 40 is shown being secured to the outer surface 16 of the chassis 12, it could alternatively be secured to the inner surface 14 of the chassis 12, if desired. However, by attaching the waistband 40 to the outer surface 16 of the chassis 12, it becomes more visually noticeable to the consumer and conveys the appearance of regular cloth underwear. This is a very desirable feature that most manufacturers are striving to accomplish. Furthermore, if desired, the waistband 40 could be integrally formed with the front and/or back regions, 18 and 20 respectively.

Referring again to FIGS. 3 and 4, the waistband 40 includes an interior surface 56 and an exterior surface 58. The interior surface 56 faces and contacts the skin of the wearer of the disposable absorbent article 10 while the outer surface 58 is spaced away from the skin of the wearer. The interior surface 56 is sometimes referred to as the body facing surface and the outer surface 58 is sometimes referred to as the garment facing surface.

Referring again to FIG. 1, the disposable absorbent article 10 further includes a removable indicium 60 positioned on the interior surface 56 of the waistband 40. By “indicium” it is meant any identifying mark, data, impression or indication made in or upon a surface. The indicium 60 can include but is not limited to a brand name, a company name, a trademark, a service mark, a logo, an icon, a graphic depiction, a symbol, artwork, one or more letters, one or more words, one or more numbers, etc. The indicium 60 further includes indicia. The indicium 60 can be formed or printed using various kinds of inks, embossed, die-cut, applied, deposited or otherwise formed as is well known to those skilled in the art. The indicium 60 can be applied or printed in black, white or in any desired color. The indicium 60 can function to provide a visual indicator for facilitating proper fitting of the disposable absorbent article 10 to a wearer's body. For example, as shown in FIG. 1, the indicium 60 can designate the inside back surface of the disposable absorbent article 10 to aid a caregiver or a wearer in positioning the article 10 correctly around the wearer's torso. Alternatively, the indicium 60 can designate the front of the disposable absorbent article 10 to aid a young child in properly stepping into the article 10. Some young children tend to look at the front of the disposable absorbent article 10 and align it with their stomach rather then searching for a back indicator or label.

Still referring to FIG. 1, one way of making the indicium 60 removable from the waistband 40 is to print it using an ink that lightens up, disappears or becomes invisible when subjected to heat, moisture, perspiration and/or humidity. A heat activated ink can be made that becomes less visible, less noticeable or totally invisible once it is maintained at normal body temperature for a predetermined amount of time. For example, inks containing thermo chromic pigments are commercially available that can lighten significantly or disappear when subjected to a certain temperature for a given period of time. In addition, inks that lighten up or disappears when subjected to moisture or perspiration can be used. By “perspiration” it is meant the saline moisture excreted through the pores of the human skin by the sweat glands. Furthermore, a humidity activated ink can become invisible or less noticeable when contacted by water or water vapor. These and other types of disappearing inks, which are well known to those skilled in the art, can also be used. Two suppliers of some of the above described inks include GBC Thermochromics having an office at Charlbury, Chipping Norton, Oxfordshire 0X7 3EW, United Kingdom and Leuco Dye Products having an office in Streamwood, Ill. 60107. Still further, the indicium 60 can be formed from glow in the dark materials or chemicals that make it easier for a user to read or see the indicium 60 in a low light or no light environment.

A more practical way of removing the indicium 60 is to form it on a removable label 62, see FIGS. 1, 3 and 5. The label 62 can be aligned with the front region 18 of the waistband 40 or be aligned with the back region 20 of the waistband 40. The label 62 can be removeably secured to the waistband 40 by an adhesive 64, see FIG. 5. The label 62 can be secured by a pressure sensitive adhesive, by a cohesive, by a hook and loop fastener or by other means known to those skilled in the art. When an adhesive 64 is used, it should exhibit a low tack and/or low peel strength so that the label 62 can be readily removed from the waistband 40. Various kinds of adhesive 64 that can be used for this application are known to those skilled in the art. When a hook and loop fastener is used, it should require a relatively low separation force to separate the hooks from the loop material.

The label 62 can be easily removed or peeled away from the waistband 40 by the wearer of the disposable absorbent article 10. The removed label 62 could be properly disposed of in a trash receptacle after it has been removed from the disposable absorbent article 10. Alternatively, the label 62 could be used to secure a previously used undergarment in a rolled up or bundle shape to provide discretion and cleanliness during disposal. Still further, the label 62 could be retained in a drawer or in some other non-conspicuous location by the user and be used at a later time to retain the soiled undergarment in a rolled up or bundle shape to ease disposal.

After the label 62 has been removed, the disposable absorbent article 10 can be pulled up along a wearer's leg and thighs and be adjusted around a wearer's torso. In FIG. 1, the indicium 60 consists of the word “BACK” which is printed on the removable label 62. The removable label 62 is positioned on the interior surface 56 of the waistband 40 above the waist opening 28. The removable label 62 is aligned with the longitudinal central axis X-X and is centered over the back region 20. Alternatively, the removable label 62 could be offset from the longitudinal central axis X-X. There are two reasons for positioning the removable label 62 on the interior surface 56 of the waistband 40. First, the label 62 can be easily seen in this location once the disposable absorbent artcile 10 is removed from its package, and second, it is easy for the consumer to remove the label 62 from this location before putting on the disposable absorbent article 10.

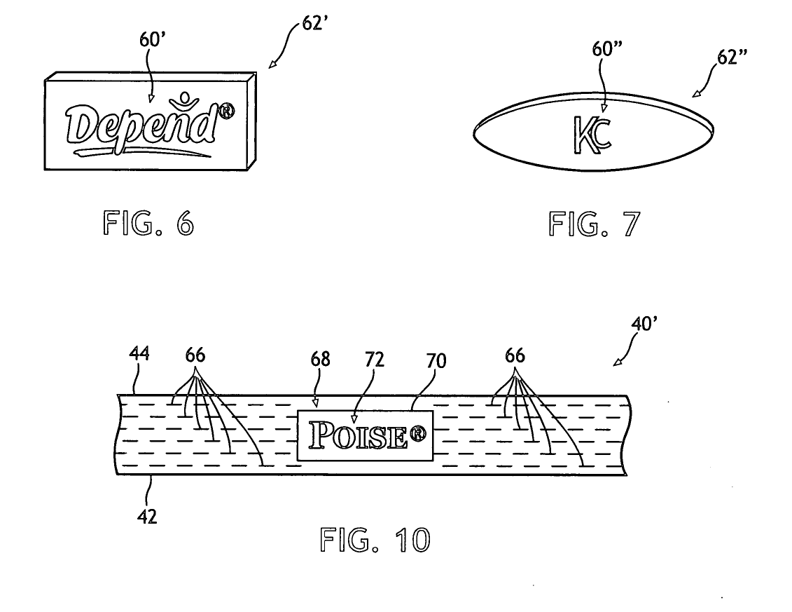

Referring now to FIG. 6, a removable label 62′ is depicted having a rectangular shape. It should be noted that any desired geometrical shape can be employed. The removable label 62′ is just one representation of an infinite variety of labels. In FIG. 6, the indicium 60′ consists of the word “DEPEND®.” The indicium 60′ is clearly visible on the major surface of the label 62′. The removable label 62′ is secured to the interior surface 56 of the waistband 40 by the manufacturer. The label 62′ functions to provide a visual indicator to the wearer in identifying the back region 20 of the disposable absorbent article 10. Since essentially all cloth underwear contains a label permanently fixed to the inside surface of the back waistband, the wearers of adult incontinent undergarments are used to seeing a label in this location. However, unlike the wearers of regular cloth underwear, most wearers of adult incontinent undergarments do not desire to have a label with the name DEPEND® printed thereon, remain on their undergarment. Psychologically, they prefer not to have the word DEPEND® printed on their incontinent undergarment even if no one else will see them wearing such products. Mental, emotional and behavioral processes can affect a person's perceptions as to how others will judge them if they find out that they are incontinent. Furthermore, when the disposable absorbent article 10 is ready to be disposed of in a trash receptacle, such as an uncovered waste basket in a bathroom, the wearer usually does not want a non-family member to quickly recognize the undergarment as being an incontinent garment.

For these reasons, the label 62′ is constructed to be easily removed by a caregiver or a wearer prior to the disposable absorbent article 10 being worn. The caregiver or wearer could quickly associate the location of the label 62′ as representing the back of the article. The label 62′ could then be easily removed before the disposable absorbent article 10 is applied to the wearer's body. The wearer can then step through the pair of leg openings 30 and 32 and pull the unitary article 10 up around his or her thighs and position the disposable absorbent article 10 about their torso. The waistband 40 will function to securely and snugly retain the disposable absorbent article 10 on the wearer's torso even after the article 10 has been insulted with a large amount of body fluid, such as urine.

Referring now to FIG. 7, an alternative removable label 62″ is depicted having an elliptical configuration. An indicium 60″ includes the letters “KC” visibly printed on a major surface of the label 62″. The letters “KC” can stand for Kimberly-Clark, the manufacturer of the disposable absorbent article 10. It should be noted that any desired letters, words, numbers, icons, etc. could be printed or otherwise formed on the label 62″. Like the removable labels 62 and 62′, the removable label 62″ can be located on the waistband 40. The label 62″ should be capable of being removed from the waistband 40 before the wearer places the disposable absorbent article 10 on their body. Alternatively, the labels 62, 62′ or 62″, even though they are removable, can be left in place if the caregiver or wearer so desires. In this regard, the labels 62, 62′ and 62″ should be constructed from a material that has a soft hand or feel. A soft material will not irritate the skin of the wearer should the caregiver or wearer decide to leave the label 62, 62′ or 62″ in place. The removable label 62, 62′ and 62″ should be constructed or formed from a soft fabric formed from a natural or synthetic material. Cotton and spunbond are two such materials. Spunbond is commercially sold by Kimberly-Clark Corporation, having an office at 401 North Lake Street, Neenah, Wis. 54956.

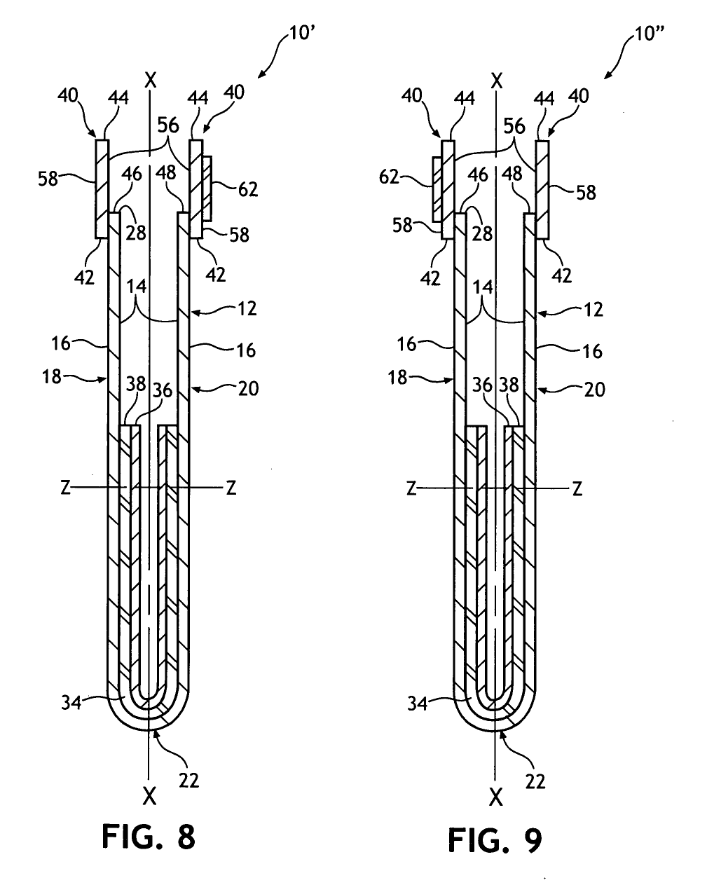

Turning now to FIGS. 8 and 9, two alternative embodiments are depicted wherein the removable label 62 is located in a different position on the waistband 40. The embodiment shown in FIG. 8 is similar to the embodiment depicted in FIG. 3, except that the removable label 62 is positioned on the exterior surface 58 of the waistband 40, above the back region 20. The removable label 62 is shown as being located above the waist opening 28 and can be aligned with or be offset from the longitudinal central axis X-X. The embodiment shown in FIG. 9 is also similar to the embodiment depicted in FIG. 3, except that the removable label 62 is positioned on the exterior surface 58 of the waistband 40, above the front region 18. The removable label 62 is again shown being located above the waist opening 28 so as to be readily visible to a caregiver or to the wearer. The label 62 can also be aligned with or be offset from the longitudinal central axis X-X. In addition, the waistband 40 can extend completely around the waist opening 28 and the pair of side seams 24 and 26 can extend up into the waistband 40. The label 62 can then be aligned with said front region 18 and can be equally spaced apart from each of the pair of side seams, 24 and 26 respectively.

It should be noted that the indicium 60 does not have to be physically attached or be positioned on a removable label 62. Instead, as mentioned above, the indicium 60 could be printed directly on the interior or exterior surfaces, 56 or 58 respectively, of the waistband 40. As the ink becomes lighter, invisible or disappears when subjected to a preconceived environmental condition, such as a given temperature value, moisture, perspiration, humidity, etc., the indicium 60 becomes less noticeable or disappears completely. 13. Referring now to FIG. 10, a length of an elastic waistband 40′ is shown having a plurality of elastic strands 66 formed therein. The elastic strands 66 do not extend over the entire length of the waistband 40′ but instead are non-continuous to create a non-elasticized area 68. Within this non-elasticized area 68 is a removable label 70 having an indicium 72 printed thereon. The indicium 72 consists of the name “POISE®.” The removable label 70 is temporarily attached to the waistband 40′. It is also possible to form the waistband 40′ such that two or more areas are void of elastics and the label 70 is attached to one of these areas.

Another way of creating the non-elasticized area 68 is to deaden the elastic strands 66 in this area. This can be accomplished by cutting or chopping the elastic strands every few millimeters in a designated area. The elastic strands 66 can also be rendered non-elastic in the area 68 by subjecting the elastic strands 66 to a high temperature, to a chemical, or to certain wave frequency. These and other ways of deadening or making the elastic strands non-stretchable in a given area are well known to those skilled in the art. One benefit of placing the removable label 70 containing the indicium 72 in a non-elastic area is that the indicium 72, as well as the label 70, will not become stretched, wrinkled or distorted by the stretching and/or retraction properties of the waistband 40′. This will assure that the printing on the indicium 72 is always readable.

It should be pointed out that the indicium 72 can be printed onto the removable label 70 before the label 70 is temporarily attached to the waistband 40′. Alternatively, the indicium 72 can be printed onto the removable label 70 after the label 70 has been attached to the waistband 40′. Still another option is to print the label 70 as it is being applied to the waist band 40′. One can print the indicium 72 using a variety of printers. It has been found that a non-impact printer, such as an ink jet printer, works well.

### METHOD

Referring again to FIG. 1, a method of promoting the sale of the disposable absorbent article 10 can be accomplished by associating the disposable absorbent article 10 with an indicium 60 that is printed on a removable label 62. The label 62 is designed to be removed before the disposable absorbent article 10 is actually worn. For example, an adult incontinent undergarment can be manufactured with a visible waistband 40 having a removable label 62 secured thereto. The removable label 62 can be attached to either the interior or to the exterior surface, 56 or 58 respectively, of the waistband 40, see FIGS. 3, 8 and 9. The removable label 62 can contain an indicium 60 indicating the name of the manufacturer, the product name, a brand name, a trademark of the manufacturer, one or more words, letters, icons, etc. The location of the removable label 62 can identify the front or the back of the disposable absorbent article 10. In this case, the indicium 60 will provide a visual cue, reminder or prompting to the wearer or to a caregiver of how to properly position or place the disposable absorbent article 10 on the wearer's body. Since the indicium 60 can be removed before the disposable absorbent article 10 is worn, the wearer will not experience any psychological effects from wearing such an undergarment.

Alternatively, the label 62 can be permanently secured to the waistband 40 such that it is not meant to be removed. In this case, the indicium 60 can be formed from an ink or other material that becomes lighter or disappears completely due to heat, temperature, light, moisture, perspiration, humidity, etc. The label 62 will assist the wearer or a caregiver in properly orienting the disposable absorbent article 10 onto the wearer's body. As the disposable absorbent article 10 is worn, the indicium 60 will become essentially invisible so that it can no longer be noticed.

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. A disposable absorbent article comprising:
a) a chassis having an inner surface and an outer surface, and having a front region and a back region joined together by a pair of side seams to form a unitary article having a waist opening and a pair of leg openings;
b) an absorbent secured to said inner surface;
c) a waistband extending along at least a portion of one of said front and back regions and aligned adjacent to said waist opening; and
d) a removable indicium positioned on said waistband which provides a visual indicator.

2. The disposable absorbent article of claim 1 wherein said waistband is secured in a cantilever fashion to said outer surface and projects upward from said waist opening.

3. The disposable absorbent article of claim 2 wherein said waistband has an interior surface which extends above said waist opening and said indicium is attached to said interior surface.

4. The disposable absorbent article of claim 2 wherein said waistband has an exterior surface extending above said waist opening and said indicium is attached to said exterior surface.

5. The disposable absorbent article of claim 2 wherein said waistband is elastic and said elastic is deadened beneath said indicium.

6. The disposable absorbent article of claim 2 wherein said indicium is printed on a label and said label is removeably attached to said waistband.

7. The disposable absorbent article of claim 6 wherein said indicium is printed on a label using a heat activated ink that will become less noticeable when maintained at a certain temperature for a predetermined period of time.

8. The disposable absorbent article of claim 1 wherein said pair of side seams extend into said waistband and said waistband extends completely around said waist opening, and said indicium is aligned with said front region and is equally spaced apart from each of said pair of side seams.

9. The disposable absorbent article of claim 1 wherein said waistband extends along at least a portion of said front region and said indicium is printed on a label aligned with said front region of said waistband.

10. A disposable absorbent article comprising:
a) a chassis having an inner surface, an outer surface, a front region, a back region and a crotch region extending between said front and back regions, said front and back regions being joined together by a pair of side seams to form a unitary article having a waist opening and a pair of leg openings;
b) an absorbent secured to said inner surface;
c) a waistband secured in a cantilever fashion to said outer surface, said waistband extending along at least a portion of said back region proximate said waist opening;
d) a label providing a visual indicator for properly fitting said article to a wearer's body, said label being removeably positioned on said waistband; and
e) an indicium printed on said label.

11. The disposable absorbent article of claim 10 wherein said waistband is elastic and said elastic is deadened beneath said label.

12. The disposable absorbent article of claim 10 wherein said indicium is printed on said label prior to said label being secured to said waistband and said indicium is printed on said label using a heat activated ink that will become less noticeable when maintained at a certain temperature for a predetermined period of time.

13. The disposable absorbent article of claim 10 wherein said waistband has an interior surface which extends above said waist opening, said interior surface having an area which is void of elastics, and said label is attached to said area that is void of elastics.

14. The disposable absorbent article of claim 10 wherein said waistband has an exterior surface extending above said waist opening and said label is attached to said exterior surface.

15. The disposable absorbent article of claim 10 wherein said waistband has an upper edge which is located from between about 0.5 inches to about 4 inches above said waist opening.

16. A disposable absorbent article comprising:
a) a chassis having an inner surface, an outer surface, a front region, a back region and a crotch region extending between said front and back regions, said front and back regions being joined together by a pair of side seams to form a unitary article having a waist opening and a pair of leg openings;
b) an absorbent secured to said inner surface;
c) an elastic waistband secured in a cantilever fashion to said outer surface, said elastic waistband secured completely around said waist opening and having an upper edge which extends at least 1 inch above said waist opening;
d) a label providing a visual indicator for properly fitting said article to a wearer's body, said label being removeably positioned on said waistband; and
e) an indicium printed on said label.

17. The disposable absorbent article of claim 16 wherein said label is removed prior to said disposable absorbent article being worn and said removed label is used to retain a previously soiled disposable absorbent article in a rolled up configuration during disposal of said previously soiled disposable absorbent article.

18. The disposable absorbent article of claim 16 wherein said indicium is printed onto said label after said label has been attached to said waistband.

19. The disposable absorbent article of claim 16 wherein said indicium is printed onto said label before said label is attached to said waistband.

20. A method of promoting the sale of a disposable absorbent article by associating said article with indicium printed on a removable label and said label is removed before said disposable absorbent article is worn.
